(19) United States
(12) Patent Application Publication
(10) Pub. No.: US 2022/0133135 A1
(43) Pub. Date: May 5, 2022

(54) FULL ROTATIONAL ARTICULATION LAPAROSCOPE

(71) Applicant: Benefsha Mohammad, Houston, TX (US)

(72) Inventor: Benefsha Mohammad, Houston, TX (US)

(21) Appl. No.: 17/519,509

(22) Filed: Nov. 4, 2021

Related U.S. Application Data
(60) Provisional application No. 63/109,728, filed on Nov. 4, 2020.

(51) Int. Cl. A61B 1/00; A61B 1/005; A61B 1/06; A61B 1/313; A61B 1/05

(52) U.S. Cl. CPC A61B 1/00147; A61B 1/0052; A61B 1/0638; A61B 1/00105; A61B 1/05; A61B 1/00009; A61B 1/00045; A61B 1/3132

(57) ABSTRACT

Laparoscopic devices, systems, and related methods of assembly and use. A laparoscopic device includes a controller connected to a camera rod having a flexible joint adjacent to a tip of the camera rod. The tip is configured to emit an incident light from a light source of the device to illuminate an anatomy of a subject and to receive light reflected from the anatomy back to the tip. The received light is processed to produce images of the anatomy that may be used during a medical procedure. The flexible joint provides a wider range of motion and a wider view angle for the tip, and the controller is more ergonomic to enable the laparoscopic device to be more easily operated.

### CROSS-REFERENCE TO RELATED APPLICATIONS

Under provisions of 35 U.S.C. § 119(e), the Applicant claims the benefit of U.S. provisional application No. 63/109,728, filed Nov. 4, 2020, which is incorporated herein by reference. It is intended that the referenced application may be applicable to the concepts and embodiments disclosed herein, even if such concepts and embodiments are disclosed in the referenced application with different limitations and configurations and described using different examples and terminology.

### STATEMENT REGARDING FEDERALLY SPONSORED RESEARCH OR DEVELOPMENT

None.

### FIELD

The disclosure relates to laparoscopic devices, systems, and related methods of assembly and use. A laparoscopic device includes a controller connected to a camera rod having a flexible joint adjacent to a tip of the camera rod. The flexible joint provides a wider range of motion and a wider view angle for the tip, and the controller is configured to be handheld and ergonomic to enable the laparoscopic device to be more easily operated.

### BACKGROUND

Existing laparoscopes may be articulated at a limited angle, for example, 30 to 40 degrees, and during use, the resultant field of view may be limited which can impact the quality of care. Many existing laparoscopes may be provided as multiple pieces, such as the camera head, the scope, and the light cord, all of which must be connected during assembly of the laparoscope. Assembly can take time and may lead to certain operational disadvantages, because a piecemeal assembly of the various parts typically does not allow for a full rotational articulation of the laparo scope.

Accordingly, there remains a need for an improved laparoscope device with endoscope functionality embedded therein and an increased rotational articulation. Additional enhancements to the laparoscope device, such as zooming capabilities with an auto focus camera for increased visibility may also be desirable. These aforementioned needs, and other needs (related to conventional laparoscopes that are not described above), are satisfied by the various aspects of the present disclosure.

### SUMMARY

To address the disadvantages associated with conventional laparoscopes, the present disclosure provides a laparoscopic device having an enhanced design that improves operation of the device and quality of care. As will be explained in detail throughout the disclosure, the disclosed laparoscope device incorporates various features, such as an embedded camera, and enhanced capabilities, such as 360-degree rotation, that are not commonly included in many conventional laparoscopes in the medical industry. The disclosed laparoscope device can also provide an endo scope functionality as a result of its design. Further, the laparoscope device, as disclosed herein, can have an ergonomic design, which allows the laparoscope device to be operated with a single hand for efficiency and comfort while being operated by a user (e.g., a surgeon). According to various embodiments, the structure of the laparoscope device may enable a maximum 360-degree articulation.

In accordance with the purposes of the present disclosure, as embodied and broadly described herein, the present disclosure, in one aspect, relates to a medical device, described herein as a laparoscope device. As alluded to above, the disclosed laparoscope includes endoscope functionality. In further aspects, the disclosed laparoscope device may have ergonomic functions which allows the device to be comfortably handled by a user, for instance a surgeon, with a single hand. In further aspects, the disclosed laparoscope device can have a camera embedded with automatic (auto) and/or manual focus and zoom in/out features. The present disclosure also describes various zooming capabilities, not limited to HD and 4K, which may enhance quality of images of an interior of a subject or patient (e.g., an interior of a subject's abdomen). As alluded to above, the disclosed laparoscope device may be structured to include an embedded camera that enables adjustment of a focal distance of the lens, a zooming feature, and a de-fogger camera lens to enable quick and easy re-framing of a scene while standing in the same physical position.

Additional aspects of the present disclosure will be set forth in part in the description which follows and which adequately sets forth, to a person having ordinary skill in the art, modes of the invention and variations thereof which are considered to be effective for the purposes intended. The advantages of the present disclosure will be realized and attained by means of elements and combinations particularly pointed out in the appended claims. It is to be understood that both the foregoing general description and the following detailed description are exemplary and explanatory only, and are not restrictive of the present disclosure, as claimed.

Accordingly, the disclosure provides a medical device in the form of a full rotational articulation laparoscope and methods and systems for laparoscopy. The laparoscope device can include a lens that is connected to a camera rod, and the lens may be configured to receive light reflected from a visible light source and/or a UV light source. The laparoscope device includes a joint that enables a tip of the camera rod to bend in a three-hundred-sixty-degree radial range. The camera rod can carry power and image signals to and/or from the camera and the light source(s). Lenses can utilize auto and/or manual zoom-in and zoom-out functionality, and auto and/or manual focus functionality, and the lenses can be prevented from becoming fogged with an automatic de-fogger. The camera rod can be implemented as a signal cable to allow for bending of the laparoscope device. The camera rod is attached to the controller, and the controller is interfaced to display the image on a display device. Also disclosed herein are methods of using the disclosed laparoscope device and related systems.

As such, the invention generally relates to laparoscopic devices and systems which may be manufactured with appropriate materials and processes and which may be scaled as needed.

Other objects, features, and advantages of the invention will become apparent from the following detailed description taken in conjunction with the accompanying drawings.

### BRIEF DESCRIPTION OF THE DRAWINGS

Although the characteristic features of the invention will be particularly pointed out in the claims, the invention itself and manners in which it may be made and used may be better understood after a review of the following description, taken in connection with the accompanying drawings, wherein like numeral annotations are provided throughout.

FIG. 1 depicts a side view of an exemplary laparo scope device, in accordance with an example of embodiment of the present disclosure.

FIG. 2A depicts a side view of an exemplary laparoscope device that includes a camera rod as an optical fiber.

FIG. 2B depicts a side view of an exemplary laparoscope device that includes a camera rod that includes a light-emitting diode (LED).

FIG. 2C depicts a perspective view of an exemplary cable (which has a connector at a distal end, shown as a Universal Serial Bus (USB) connector) that can connect the laparoscope device to a computer and/or a display device.

FIG. 3A depicts a side view of an exemplary laparoscope system that includes a laparoscope device connected to a computer.

FIG. 3B depicts a side view of an exemplary laparoscope system that includes a laparoscope device connected to a display.

FIG. 4 depicts a side view of several ergonomic features of an exemplary laparoscopic device.

FIG. 5A depicts a rear view of a first exemplary controller of a laparoscope device.

FIG. 5B depicts a rear view of a second exemplary controller of a laparoscope device.

FIG. 5C depicts a rear view of a third exemplary controller of a laparoscope device.

FIG. 6 depicts a block diagram of a machine in the example form of a computer system.

FIG. 7A depicts a cross-section of exemplary mechanism(s) that may be incorporated into a laparoscopic device to control movement of the tip.

FIG. 7B depicts a perspective view of exemplary mechanism(s) that may be incorporated into a laparoscopic device to control movement of the tip.

FIG. 7C depicts a perspective view of exemplary mechanism(s) that may be incorporated into a laparoscopic device to control movement of the tip.

FIG. 7D depicts a side view of exemplary mechanism(s) that may be incorporated into a laparoscopic device to control movement of the tip.

FIG. 7E depicts side breakaway views of exemplary mechanism(s) that may be incorporated into a laparoscopic device to control movement of the tip.

FIG. 7F depicts a side view of exemplary mechanism(s) that may be incorporated into a laparoscopic device to control movement of the tip, with broken lines indicating positions of cross sections for FIGS. 7G, 7H, 7I, 7J, and 7K.

FIG. 7G depicts a cross section of exemplary mechanism(s) that may be incorporated into a laparoscopic device to control movement of the tip.

FIG. 7H depicts a cross section of exemplary mechanism(s) that may be incorporated into a laparoscopic device to control movement of the tip.

FIG. 7I depicts a cross section of exemplary mechanism(s) that may be incorporated into a laparoscopic device to control movement of the tip.

FIG. 7J depicts a cross section of exemplary mechanism(s) that may be incorporated into a laparoscopic device to control movement of the tip.

FIG. 7K depicts a cross section of exemplary mechanism(s) that may be incorporated into a laparoscopic device to control movement of the tip.

FIG. 7L depicts a side breakaway view of exemplary mechanism(s) that may be incorporated into a laparoscopic device to control movement of the tip.

FIG. 7M depicts side breakaway views of exemplary mechanism(s) that may be incorporated into a laparoscopic device to control movement of the tip.

FIG. 7N depicts side breakaway views of exemplary mechanism(s) that may be incorporated into a laparoscopic device to control movement of the tip.

FIG. 7O depicts side breakaway views of exemplary mechanism(s) that may be incorporated into a laparoscopic device to control movement of the tip.

FIG. 7P depicts side breakaway views of exemplary mechanism(s) that may be incorporated into a laparoscopic device to control movement of the tip.

FIG. 7Q depicts a side view of exemplary mechanism(s) that may be incorporated into a laparoscopic device to control movement of the tip.

FIG. 7R depicts a side view of exemplary mechanism(s) that may be incorporated into a laparoscopic device to control movement of the tip.

FIG. 7S depicts a side view of exemplary mechanism(s) that may be incorporated into a laparoscopic device to control movement of the tip.

FIG. 7T depicts a side view (top) and close-up views (bottom) of exemplary mechanism(s) that may be incorporated into a laparoscopic device to control movement of the tip.

### DETAILED DESCRIPTION OF THE INVENTION

Reference is made herein to the attached drawings. Like reference numerals may be used in the drawings to indicate like or similar elements of the description. The figures are intended for representative purposes and should not be considered limiting.

The present disclosure can be understood more readily by reference to the following detailed description of the present disclosure and the examples included therein.

Before the present articles, systems, devices, and/or methods are disclosed and described, it is to be understood that they are not limited to specific manufacturing methods unless otherwise specified, or to particular materials unless otherwise specified, as such can, of course, vary. It is also to be understood that the terminology used herein is for the purpose of describing aspects only and is not intended to be limiting. Although any methods and materials similar or equivalent to those described herein can be used in the practice or testing of the present disclosure, example methods and materials are now described.

All publications mentioned herein are incorporated herein by reference to disclose and describe the methods and/or materials in connection with which the publications are cited.

### Definitions

It is to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this present disclosure belongs. In this specification and in the claims which follow, reference will be made to a number of terms which shall be defined herein.

As used in the specification and the appended claims, the singular forms “a,” “an,” and “the” include plural referents unless the context clearly dictates otherwise. Thus, for example, reference to “an opening” can include two or more openings.

Ranges can be expressed herein as from one particular value, and/or to another particular value. When such a range is expressed, another aspect includes from the one particular value and/or to the other particular value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

The terms “first,” “second,” “first part,” “second part,” and the like, where used herein, do not denote any order, quantity, or importance, and are used to distinguish one element from another, unless specifically stated otherwise.

As used herein, the terms “optional” or “optionally” means that the subsequently described event or circumstance can or cannot occur, and that the description includes instances where said event or circumstance occurs and instances where it does not. For example, the phrase “optionally affixed to the surface” means that it can or cannot be fixed to a surface.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including matters of logic with respect to arrangement of steps or operational flow; plain meaning derived from grammatical organization or punctuation; and the number or type of aspects described in the specification.

Disclosed are the components to be used to manufacture the disclosed devices, systems, and articles of the present disclosure as well as the devices themselves to be used within the methods disclosed herein. These and other materials are disclosed herein, and it is understood that when combinations, subsets, interactions, groups, etc. of these materials are disclosed that while specific reference of each various individual and collective combinations and permutation of these materials cannot be explicitly disclosed, each is specifically contemplated and described herein. For example, if a particular material is disclosed and discussed and a number of modifications that can be made to the materials are discussed, specifically contemplated is each and every combination and permutation of the material and the modifications that are possible unless specifically indicated to the contrary. Thus, if a class of materials A, B, and C are disclosed as well as a class of materials D, E, and F and an example of a combination material, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the articles and devices of the present disclosure. Thus, if there are a variety of additional steps that can be performed it is understood that each of these additional steps can be performed with any specific aspect or combination of aspects of the methods of the present disclosure.

It is understood that the devices and systems disclosed herein have certain functions. Disclosed herein are certain structural requirements for performing the disclosed functions, and it is understood that there are a variety of structures that can perform the same function that are related to the disclosed structures, and that these structures will typically achieve the same result.

Laparoscope Devices and Systems

In accordance with the disclosure, the structure of the laparoscope device can be described as ergonomic. Also, embodiments of the laparoscope device may be implemented by including endoscope capabilities within the laparoscope device, allowing the disclosed laparoscope device to have dual and/or multiple functions (e.g., endoscope and laparoscope functionality together in a single device). The laparoscope device may be employed in various practical applications serving in any medical field, including but not limited to intra-operative surgical activities, diagnostic procedures, biopsy capture, exploratory procedures, placement of surgical or medical implants, and the like.

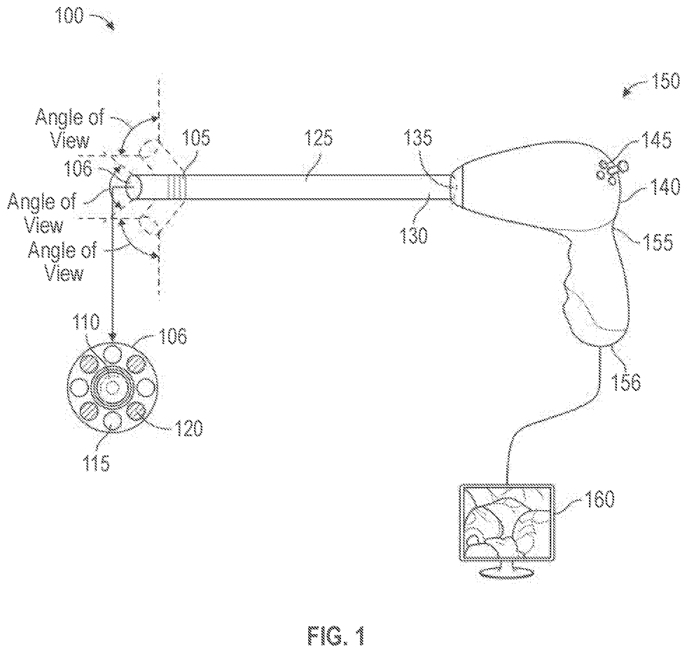

Referring now to FIG. 1, there is depicted a side view of an exemplary laparoscope device, in accordance with an example of embodiment of the present disclosure. In general, the disclosure provides a system 100 having a device 150 for laparoscopy, the device 150 comprising a controller 155 configured as an ergonomic handle, and a camera rod 125 attachable to the controller 155 and having a flexible joint 105 adjacent to a tip 106 thereof. The flexible joint 105 provides a range of motion to the tip 106, and the tip 106 is configured to emit an incident light to illuminate an anatomy of a subject and receive a reflected light to image the anatomy of the subject. In various implementations, the incident light and the reflected light comprises visible light (emitted from visible light source(s) 115), ultraviolet (UV) light (emitted from UV light source(s) 120), or both the visible light and the UV light.

In various implementations, the range of motion of the tip 106 is up to and including a maximum 360-degree range of motion and each of a pitch and a yaw of the tip 106 of the camera rod 125 includes the maximum 360-degree range of motion. An articulation of the camera rod 125 by the controller 155 moves the tip 106 about the flexible joint 105 for a pitch movement, a yaw movement, or both the pitch movement and the yaw movement. In various implementations, the device 150 may be operably connected to a display device 160 for display of an image of an anatomy during a medical procedure. In various implementations, as shown in a close-up frontal view of an end face of the tip 106 as shown in FIG. 1, includes a lens 110; and light source(s) such as visible light source(s) 115, and/or UV light source(s) 120, or any combination thereof. Incident light is emitted from the light source(s) where it strikes the anatomy of the subject and is scattered and reflected back to the lens, which focuses the reflected light back to a light sensor positioned either at the tip or at the controller.

In various implementations, a second portion 135 of the camera rod 125 (at a distal end with respect to the tip 106) may be detachable from the controller 155 such that the camera rod 125 may be detached from the controller 155, however, in other implementations, the camera rod 125 may not be detachable, such that the laparoscope device 150 has a partially monolithic design (e.g., one single unit with the camera rod and camera head being a single continuous device). A portion of the laparoscope device 150 can be detachable from various electric connectors and the controller 155, for instance, an interface 156 to a display 160 may carry data of the image and the display 160 may show the image. In various implementations, a switch panel 145 may be connected to the camera rod 125 and a main power source 140 (e.g., an internal power source disposed within the controller 155). As shown, the controller 155 may be interfaced with the display device 160, which may be a monitor or a computer device. The display device 160 may be configured to display images captured by the laparoscope device 150.

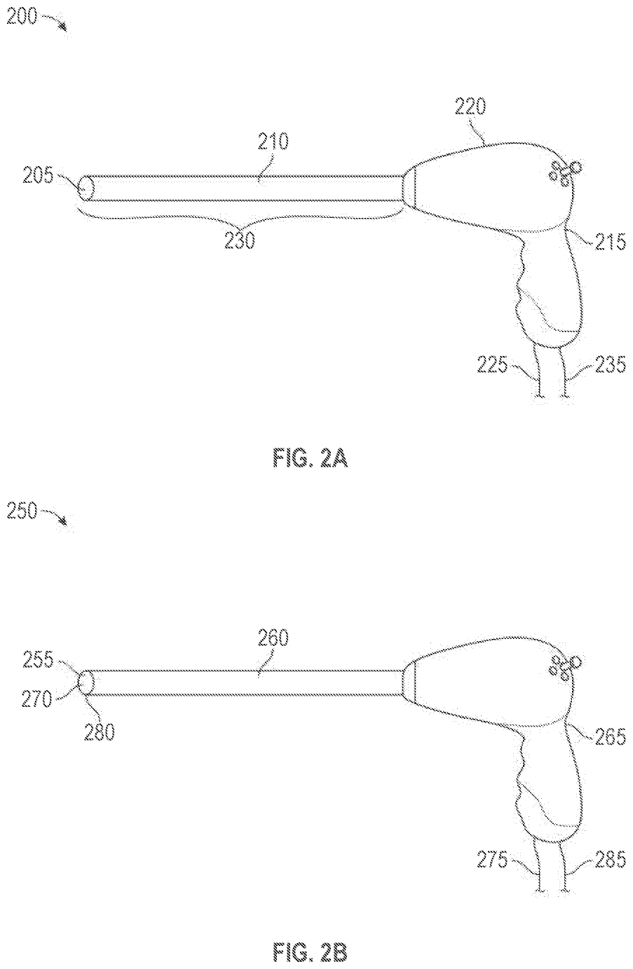

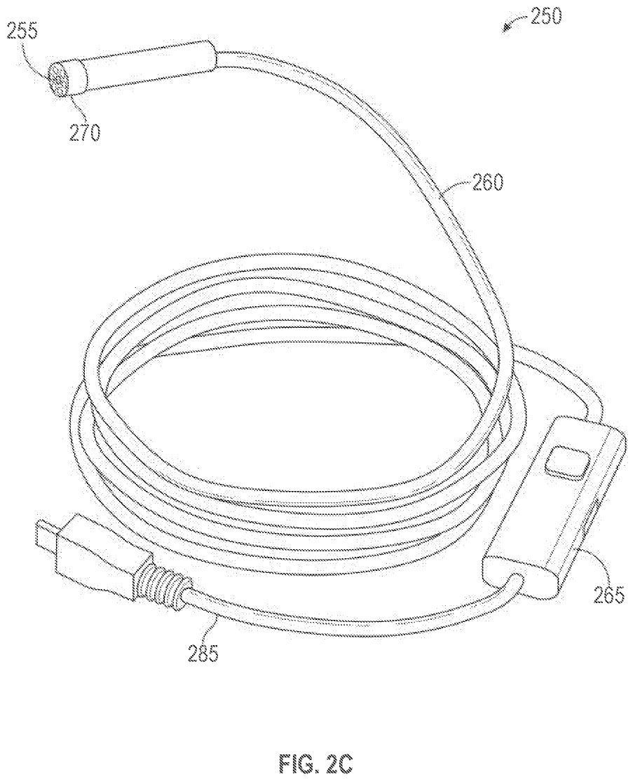

Referring now to FIG. 2A, there is depicted a side view of an exemplary laparoscope device that includes a camera rod as an optical fiber. In the shown implementation, the tip comprises a lens 205 and the camera rod 210 houses at least one optical fiber which, when combined with an image sensor 220 (e.g., an image sensor internal to the controller 215), forms a camera 230. The lens 205 and the optical fiber 210 are configured to transmit the reflected light from the anatomy to the image sensor 220, and the image sensor 220 is configured to convert the reflected light to an electrical signal that is transmitted to a processor associated with the device to convert the electrical signal to an image for display. The processor may be integral with the laparoscope device and/or may be an external processor implemented as a component of a computer system. The laparoscope device 200 may include a cable 225 that connects to a main power source (not shown) and/or a cable 235 that connects to a display device (not shown).

Referring now to FIG. 2B, there is depicted a side view of an exemplary laparoscope device that includes a camera rod that includes a light-emitting diode (LED). In the shown implementation, the camera rod houses at least one signal cable 260 having a lens 255 and an image sensor 270 at the tip of the camera rod. In this configuration, the camera 280 (combination of the lens 255 and the image sensor 270) can be described as being implemented at the tip of the laparoscope device 250. This configuration may be optimal for applications that require flexible manipulation of the device 250. For instance, contrary to certain optical fibers, a signal cable 260 can allow for greater bending, which may be used to navigate the laparoscope device 250 into a position for sufficient view of a particular organ in the abdomen of a patient. The lens 255 and the image sensor 270 are configured to convert the reflected light to an electrical signal and the signal cable 260 is configured to transmit the electrical signal to a processor associated with the device to convert the electrical signal to an image for display. The processor may be integral with the laparoscope device and/or may be an external processor implemented as a component of a computer system. The laparoscope device 250 may include a cable 275 that connects to a main power source (not shown) and/or a cable 285 that connects to a display device (not shown).

Referring now to FIG. 2C, there is depicted a perspective view of an exemplary cable (which has a connector at a distal end, shown as a Universal Serial Bus (USB) connector) that can connect the laparoscope device to a computer and/or a display device. The laparoscope device 250 (also shown in FIG. 2B) may be implemented as a signal cable 260. The tip of the signal cable 260 includes both the lens 255 and the image sensor 270 (also referred to as the camera), as illustrated. Thus, the signal cable 260 can carry the electric image signal from the tip of the signal cable 260 to the controller 265. Additionally, FIG. 2C shows a cable 285 that can connect the laparoscope device 250 to a display device (not shown). In this example, the cable 285 has a connector at a distal end, shown as a Universal Serial Bus (USB) connector, that can be plugged into a port of the display device to establish the connection.

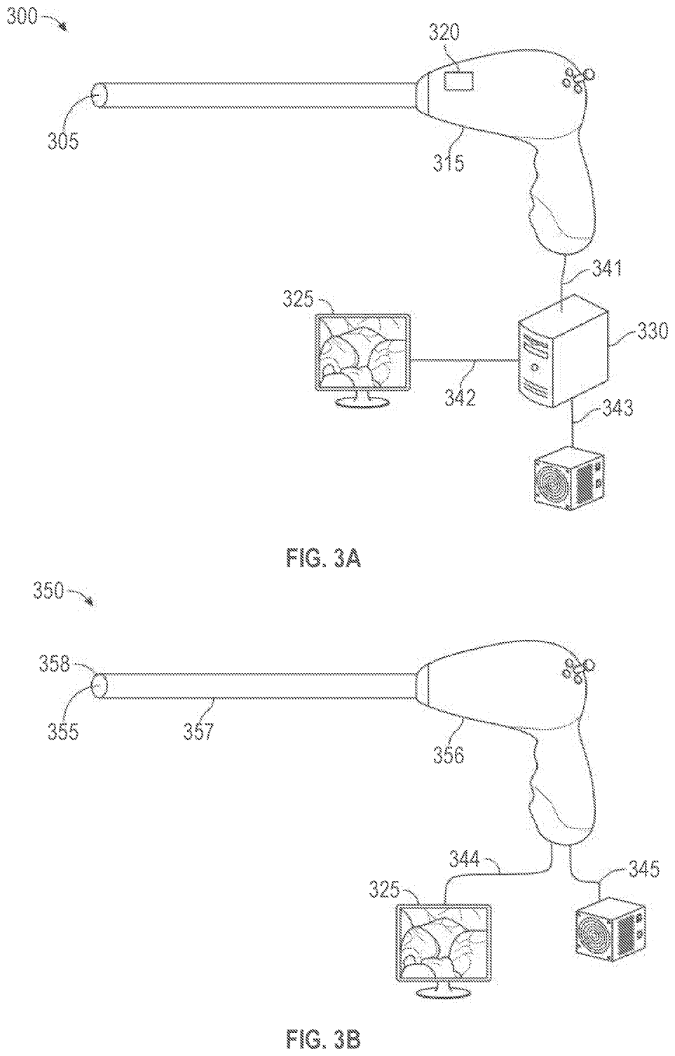

Referring now to FIGS. 3A and 3B, there are depicted a side view of an exemplary laparoscope system that includes a laparoscope device connected to a computer (FIG. 3B) and a side view of an exemplary laparoscope system that includes a laparoscope device connected to a display (FIG. 3C). These implementations provide a system for laparoscopy, the system comprising a device (300, 350) for laparoscopy, the device (300, 350) comprising a controller (315, 356) configured as an ergonomic handle, and a camera rod attachable to the controller (315, 356) and having a flexible joint adjacent to a tip thereof. The camera rod may be implemented as a signal cable 357, as shown. The flexible joint provides a range of motion to the tip, and the tip is configured to emit an incident light to illuminate an anatomy of a subject and receive a reflected light to image the anatomy of the subject. The device (300, 350) includes an image sensor 320 configured to receive the reflected light and transmit an electrical signal based on the reflected light to a processor.

Accordingly, in various implementations, the system includes the processor and a non-transitory machine-readable medium having instructions stored thereon which, when executed by the processor, configure the processor to receive the electrical signal and transmit an image signal to a display 325 configured to depict an image of the anatomy of the subject based on the image signal. The processor and the non-transitory machine-readable medium may be disposed within the device 300 (as in FIG. 3B) or may be disposed within the computer 330 (as in FIG. 3A). The computer 330 (FIG. 3A) and/or the device 300 (FIG. 3B) may be connected to an external power source via a suitable power cable (343, 345) and the device 300 may be connected to the display 325 via an image cable 344, as shown in FIG. 3B, or may be connected to the display 325 via the computer 330, as shown in FIG. 3A. The power cable (343, 345) is configured for transfer of electrical power for powering the computer 330 and peripherals (e.g., mouse, keyboard, display 325, etc.) (FIG. 3A) and/or for powering the device (300, 350) (FIG. 3B) for displaying image data on a screen of the display 325. In various implementations, the display is integral with the controller (as described elsewhere herein), but in addition or in the alternative, the display is integral with or operably connectable to the computer 330.

In various implementations, the laparoscope device includes two cables that connect to one tower. One cable carries the light source, and the other cable connects to the same tower and serves as the power source.

In various implementations, the incident light and the reflected light comprises visible light, ultraviolet (UV) light, or both. The range of motion is a maximum 360-degree range of motion of a pitch and a yaw of the tip of the camera rod, and an articulation of the camera rod by the controller moves the tip about the flexible joint for a pitch movement, a yaw movement, or both. In addition, the tip may comprise a lens (305, 355) and the camera rod be in the form of an optical fiber, and the lens (305, 355) and the optical fiber are configured to transmit the reflected light from the anatomy to the image sensor 320. In various implementations, the camera rod houses at least one signal cable 357 having a lens 355 and the image sensor 358 is positioned at the tip of the camera rod, such that the lens 355 and the image sensor 358 are configured to convert the reflected light to the electrical signal and the signal cable 357 is configured to transmit the electrical signal to the processor.

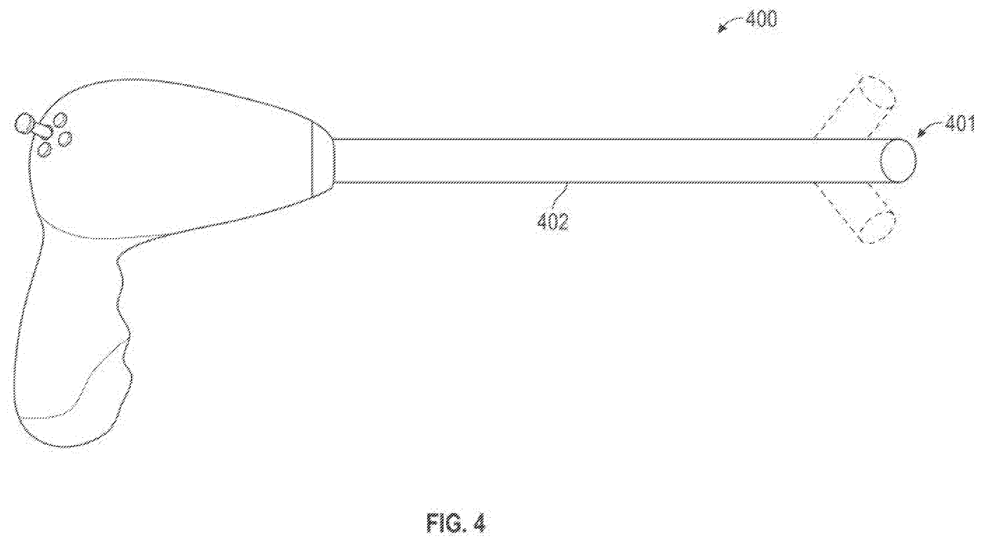

Referring now to FIG. 4, there is depicted a side view of several ergonomic features of an exemplary laparoscopic device 400. The lens 401 may be attached to the camera rod 402, which may allow for the camera to be operated with single hand in a manner that allows the operator's hand to rest. It should be appreciated that the configuration shown in FIG. 4 serves as an illustrative example and is not intended to be limiting.

In implementations, as shown, the ergonomic features include that the handle can be held in one hand, and that the articulation of the tip and the zooming in and out of the camera can all be done with one hand rather than two hands as is the case using existing laparoscopes.

In implementations, as shown, the control panel or touch screen or buttons to control movement of the tip as well zoom features are placed in a strategic position at the 12′o clock position that can be easily controlled the thumb in the vertical position. The position would be a in location that would make them to be under the thumb and the camera head is held. This makes it ergonomic and intuitive in using the buttons.

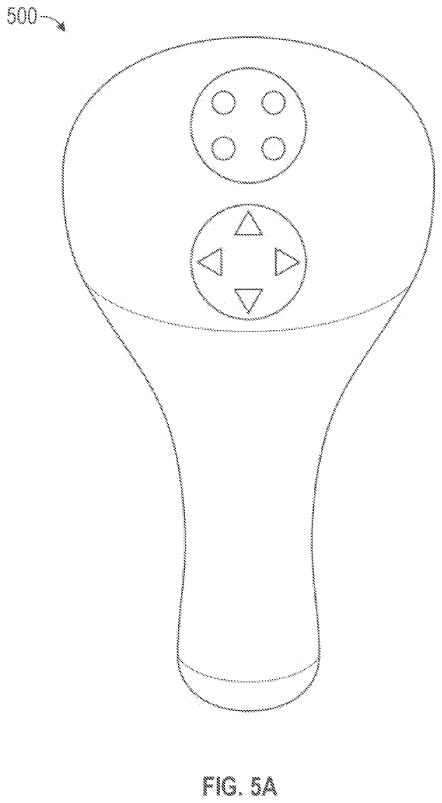

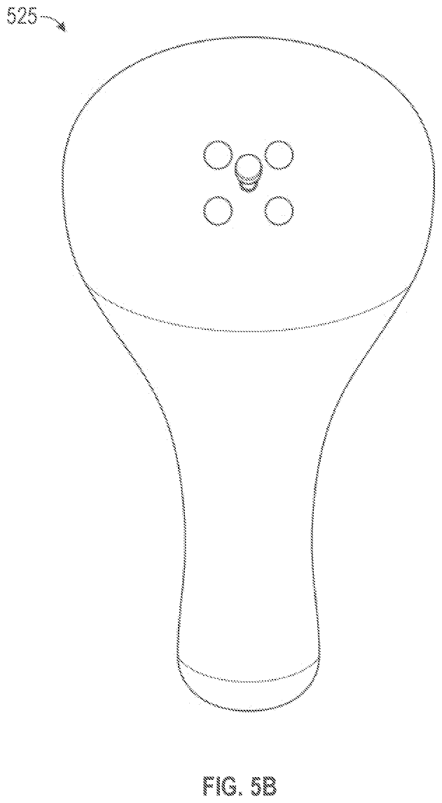

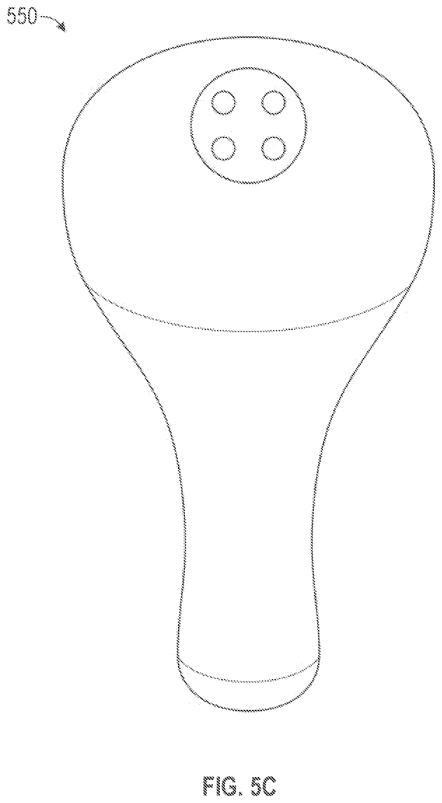

Referring now to FIGS. 5A, 5B, and 5C, there are depicted a rear view of a first exemplary controller (FIG. 5A), a rear view of a second exemplary controller (FIG. 5B), and a rear view of a third exemplary controller (FIG. 5C) of a laparoscope device. In general, the disclosure provides a controller (500, 525, 550) for a device for laparoscopy, the controller comprising an ergonomic handle configured to be grasped single-handedly, a mechanism associated with the ergonomic handle and configured to control a maximum 360-degree range of motion of a pitch and a yaw of a tip of a camera rod, and a switch panel configured to be operated single-handedly. The switch panel is operably connected to the mechanism to control the pitch and the yaw of the tip of the camera rod with movement of the mechanism.

In implementations, the controller further comprises a display that is integral with the controller, positioned adjacent to the switch panel, and configured to depict images of an anatomy of a subject thereon. In various implementations, the tip comprises a lens and the camera rod houses at least one optical fiber, and the lens and the optical fiber are configured to transmit the reflected light from an anatomy of a subject to an image sensor associated with the device, and the image sensor is configured to convert the reflected light to an electrical signal that is transmitted to a processor associated with the device to convert the electrical signal to an image for display.

The control panel can be a series of buttons with a central “joystick” button or can be a switch panel of 4 push buttons. The buttons can be implemented as a touch screen rather than or in addition to physical buttons.

In various implementations, a central button, when pushed down, can pull up a main menu of a user interface. The menu would enable an operator to control insufflation (turn on CO2 at the control tower where the light source and all other equipment are connected), control white balancing of an image depicted by the device or system, control the light source and turn it on and off, control zoom features, etc. These features can be easily controlled with one hand of the operator. In various implementations, in the diamond shape configuration, the up and down buttons can be used for operating and navigating the menu. The left and right buttons may enable the operator to zoom in and out without going to the main menu.

In various implementations, a camera rod that may be used with the controller (500, 525, 550) houses at least one signal cable having a lens and an image sensor at the tip of the camera rod, wherein the lens and the image sensor are configured to convert the reflected light to an electrical signal and the signal cable is configured to transmit the electrical signal to a processor associated with the device to convert the electrical signal to an image for display.

Autofocus

In various implementations, the camera is configured to provide an autofocus functionality and emit images in HD or 4K format. This provides additional convenience to the operator during use.

Viewing Angle

Existing laparoscopes have various sizes such as 3 mm, 5 mm, or 10 mm, and the presently disclosed laparoscope devices can be any size, however, in various implementations, the tip of the lens may be spherical to allow for a wider-angle view.

The operations, algorithms, and methods of the present invention may generally be implemented in suitable combinations of software, hardware, firmware, or a combination thereof, and the provided functionality may be grouped into a number of components, modules, or mechanisms. Modules can constitute either software modules (e.g., code embodied on a non-transitory machine-readable medium) or hardware-implemented modules. A hardware-implemented module is a tangible unit capable of performing certain operations and can be configured or arranged in a certain manner. In example embodiments, one or more computer systems (e.g., a standalone, client, or server computer system) or one or more processors can be configured by software (e.g., an application or application portion) as a hardware-implemented module that operates to perform certain operations as described herein.

In embodiments, a hardware-implemented module can be implemented mechanically or electronically. For example, a hardware-implemented module can comprise dedicated circuitry or logic that is permanently configured (e.g., as a special-purpose processor, such as a field programmable gate array (FPGA) or an application-specific integrated circuit (ASIC)) to perform certain operations. A hardware-implemented module can also comprise programmable logic or circuitry (e.g., as encompassed within a general-purpose processor or other programmable processor) that is temporarily configured by software to perform certain operations. It will be appreciated that the decision to implement a hardware-implemented module mechanically, in dedicated and permanently configured circuitry, or in temporarily configured circuitry (e.g., configured by software) can be driven by cost and time considerations.

Accordingly, the term “hardware-implemented module” should be understood to encompass a tangible entity, be that an entity that is physically constructed, permanently configured (e.g., hardwired), or temporarily or transitorily configured (e.g., programmed) to operate in a certain manner, to perform certain operations described herein, or both. Considering embodiments in which hardware-implemented modules are temporarily configured (e.g., programmed), each of the hardware-implemented modules need not be configured or instantiated at any one instance in time. For example, where the hardware-implemented modules comprise a general-purpose processor configured using software, the general-purpose processor can be configured as respective different hardware-implemented modules at different times. Software can accordingly configure a processor, for example, to constitute a particular hardware-implemented module at one instance of time and to constitute a different hardware-implemented module at a different instance of time.

Hardware-implemented modules can provide information to, and receive information from, other hardware-implemented modules. Accordingly, the described hardware-implemented modules can be regarded as being communicatively coupled. Where multiple such hardware-implemented modules exist contemporaneously, communications can be achieved through signal transmission (e.g., over appropriate circuits and buses that connect the hardware-implemented modules). In embodiments in which multiple hardware-implemented modules are configured or instantiated at different times, communications between such hardware-implemented modules can be achieved, for example, through the storage and retrieval of information in memory structures to which the multiple hardware-implemented modules have access. For example, one hardware-implemented module can perform an operation and store the output of that operation in a memory device to which it is communicatively coupled. A further hardware-implemented module can then, at a later time, access the memory device to retrieve and process the stored output. Hardware-implemented modules can also initiate communications with input or output devices, and can operate on a resource (e.g., a collection of information).

The various operations of example methods described herein can be performed, at least partially, by one or more processors that are temporarily configured (e.g., by software) or permanently configured to perform the relevant operations. Whether temporarily or permanently configured, such processors can constitute processor-implemented modules that operate to perform one or more operations or functions. The modules referred to herein can, in some example embodiments, comprise processor-implemented modules.

Similarly, the methods described herein can be at least partially processor-implemented. For example, at least some of the operations of a method can be performed by one of processors or processor-implemented modules. The performance of certain of the operations can be distributed among the one or more processors, not only residing within a single machine, but deployed across a number of machines. In embodiments, the processor or processors can be located in a single location (e.g., within an office environment, or a server farm), while in other embodiments the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), these operations being accessible via a network (e.g., the Internet) and via one or more appropriate interfaces (e.g., application program interfaces (APIs)).

Example embodiments can be implemented in digital electronic circuitry, in computer hardware, firmware, or software, or in combinations thereof. Example embodiments can be implemented using a computer program product, e.g., a computer program tangibly embodied in an information carrier, e.g., in a machine-readable medium for execution by, or to control the operation of, data processing apparatus, e.g., a programmable processor, a computer, or multiple computers.

A computer program can be written in any form of description language, including compiled or interpreted languages, and it can be deployed in any form, including as a standalone program or as a module, subroutine, or other unit suitable for use in a computing environment. A computer program can be deployed to be executed on one computer or on multiple computers at one site or distributed across multiple sites and interconnected by a communication network.

In example embodiments, operations can be performed by one or more programmable processors executing a computer program to perform functions by operating on input data and generating output. Method operations can also be performed by, and apparatus of example embodiments can be implemented as, special purpose logic circuitry, e.g., an FPGA or an ASIC.

The computing system can include clients and servers. A client and server are generally remote from each other and typically interact through a communication network. The relationship of client and server arises by virtue of computer programs running on the respective computers and having a client-server relationship to each other. In embodiments deploying a programmable computing system, it will be appreciated that both hardware and software architectures merit consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine) and software architectures that can be deployed, in various example embodiments.

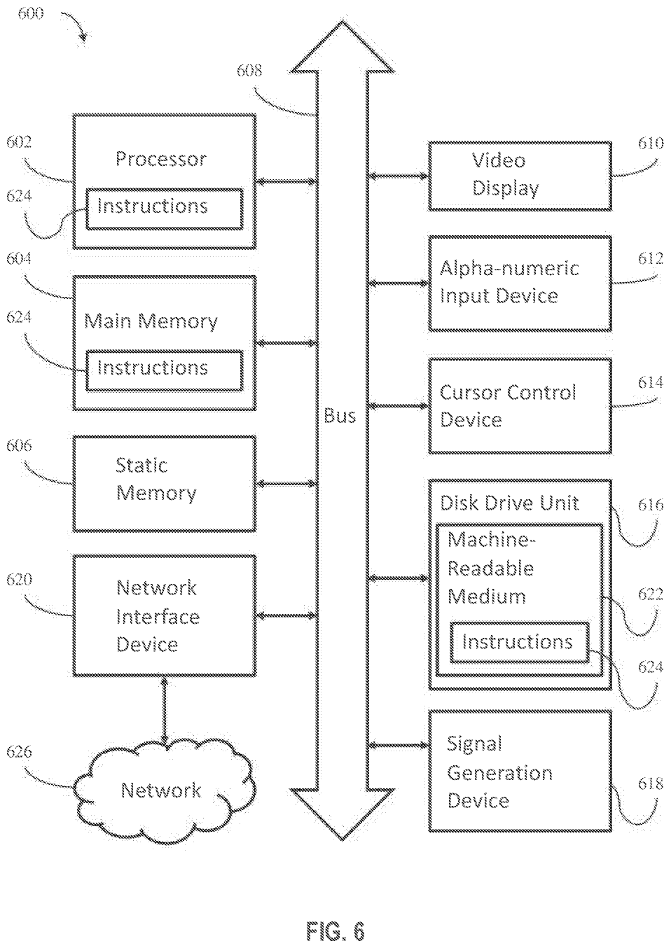

Referring now to FIG. 6, there is depicted a block diagram of a machine in the example form of a computer system 600 within which instructions 624 may be executed to cause the machine to perform any one or more of the methodologies discussed herein. The computer system 600 may include and/or be operably connected to the display 160 (FIG. 1), the display 325 (FIG. 3A and/or FIG. 3B), or any combination thereof. The computer system 600 may be implemented in whole or in part as a component of any device or system disclosed herein.

In embodiments, the machine operates as a standalone device or can be connected (e.g., networked) to other machines. In a networked deployment, the machine can operate in the capacity of a server or a client machine in server-client network environment, or as a peer machine in a peer-to-peer (or distributed) network environment. The machine can be a personal computer (PC), a tablet PC, a set-top box (STB), a personal digital assistant (PDA), a cellular telephone, a web appliance, a network router, switch, or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 600 includes a processor 602 (e.g., a central processing unit (CPU), a graphics processing unit (GPU), or both), a main memory 604, and a static memory 606, which communicate with each other via a bus 608. The computer system 600 can further include a video display 610 (e.g., a liquid crystal display (LCD) or a cathode ray tube (CRT)). The computer system 600 also includes an alpha-numeric input device 612 (e.g., a keyboard or a touch-sensitive display screen), a user interface (UI) navigation (or cursor control) device 614 (e.g., a mouse), a disk drive unit 616, a signal generation device 618 (e.g., a speaker), and a network interface device 620.

The disk drive unit 616 includes a machine-readable medium 622 on which are stored one or more sets of data structures and instructions 624 (e.g., software) embodying or utilized by any one or more of the methodologies or functions described herein. The instructions 624 can also reside, completely or at least partially, within the main memory 604 or within the processor 602, or both, during execution thereof by the computer system 600, with the main memory 604 and the processor 602 also constituting machine-readable media.

While the machine-readable medium 622 is shown in an example embodiment to be a single medium, the term “machine-readable medium” can include a single medium or multiple media (e.g., a centralized or distributed database, and/or associated caches and servers) that store the one or more instructions 624 or data structures. The term “machine-readable medium” shall also be taken to include any tangible medium that is capable of storing, encoding, or carrying instructions 624 for execution by the machine and that cause the machine to perform any one or more of the methodologies of the present disclosure, or that is capable of storing, encoding, or carrying data structures utilized by or associated with such instructions 624. The term “machine-readable medium” shall accordingly be taken to include, but not be limited to, solid-state memories, and optical and magnetic media. Specific examples of machine-readable media 622 include non-volatile memory, including by way of example semiconductor memory devices, e.g., erasable programmable read-only memory (EPROM), electrically erasable programmable read-only memory (EEPROM), and flash memory devices; magnetic disks such as internal hard disks and removable disks; magneto-optical disks; and CD-ROM and DVD-ROM disks.

The instructions 624 can be transmitted or received over a communication network 626 using a transmission medium. The instructions 624 can be transmitted using the network interface device 620 and any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi and WiMax networks). The term “transmission medium” shall be taken to include any intangible medium that is capable of storing, encoding, or carrying instructions 624 for execution by the machine, and includes digital or analog communications signals or other intangible media to facilitate communication of such software.

Methods for Using the Laparoscope Device and System

A method is disclosed for using a laparoscope device of the present disclosure that includes gripping the laparoscope device with one hand and operating the laparoscope device with the one hand. The method may prevent the operator from needing to operate the laparoscope device with another hand, whether a second hand of the operator or a hand of another individual. By using the improved and ergonomic laparoscope device, performing laparoscopy is more efficient and the operator can hold the device in one hand for better mobility and control the device easily with the one hand to view an anatomy of a subject or patient, such as internal organs of the abdomen of the subject or the patient, during a laparoscopy procedure and/or a minimally invasive surgical procedure.

Mechanism

The mechanism which operably connects the switch panel to the tip of the camera rod may be implemented with any of a variety of mechanisms, including but not limited to any one or more mechanisms as described herein. The mechanism enables the switch panel to control the pitch and the yaw of the tip of the camera rod with movement of the mechanism which may be electronical, automatic, semi-automatic, mechanical, or manual.

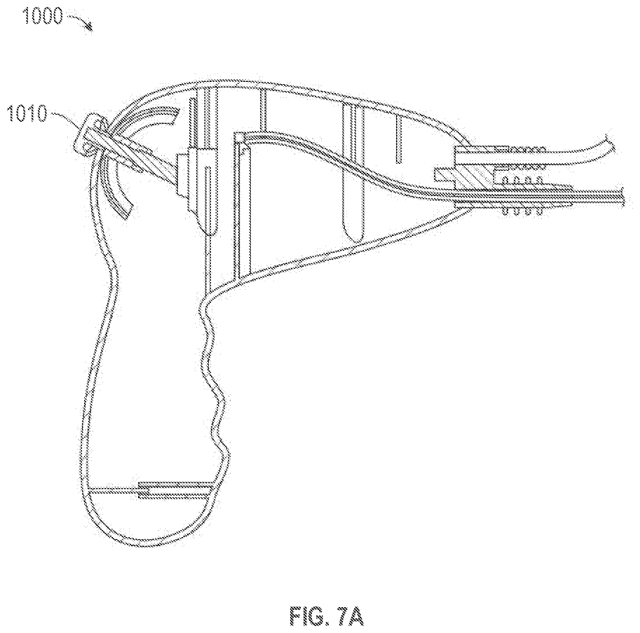

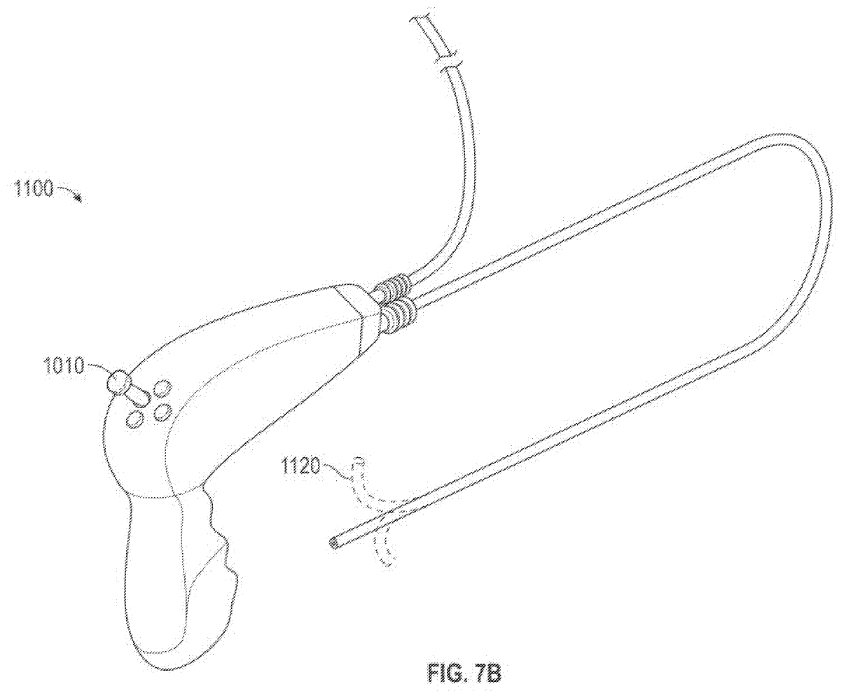

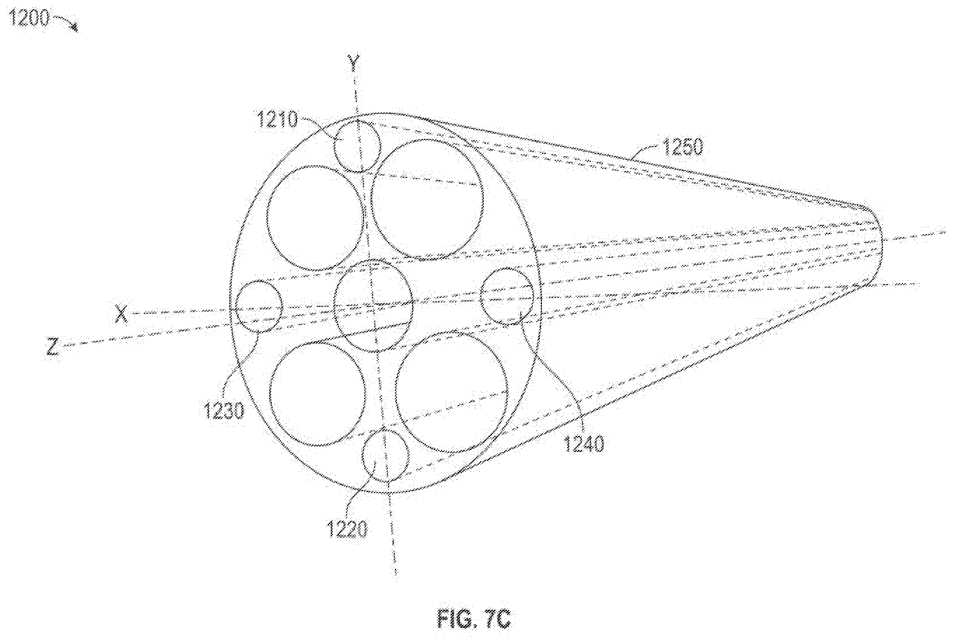

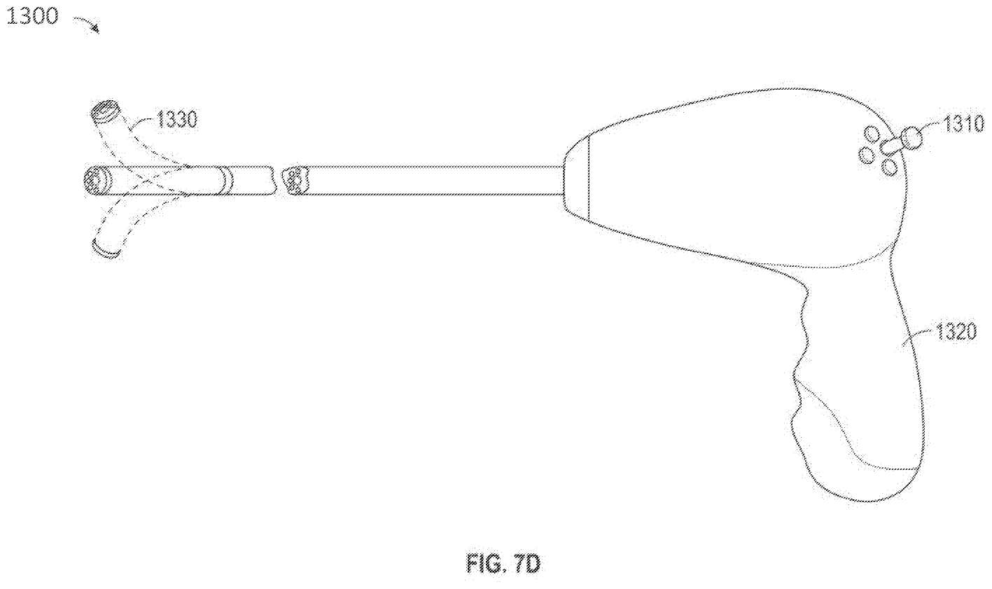

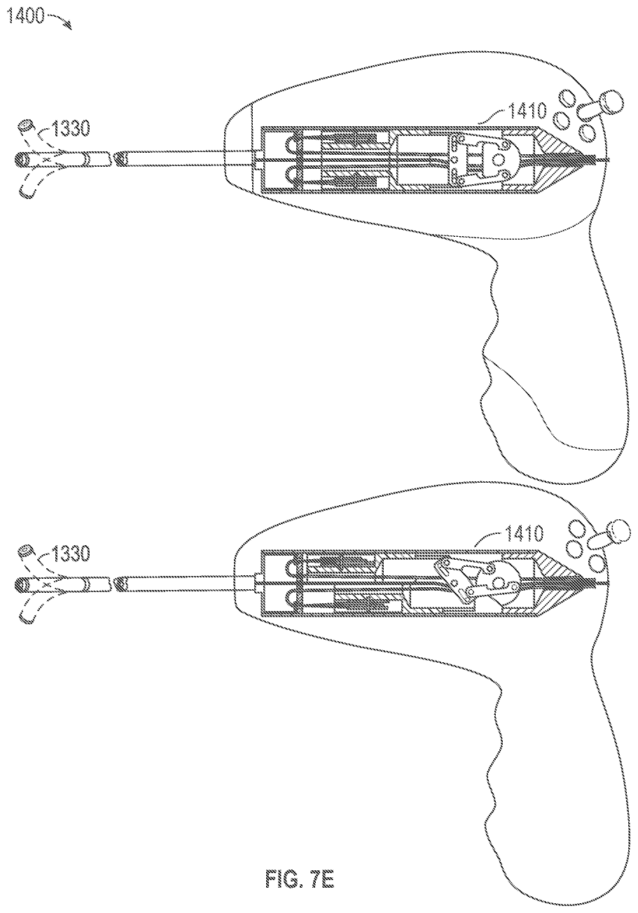

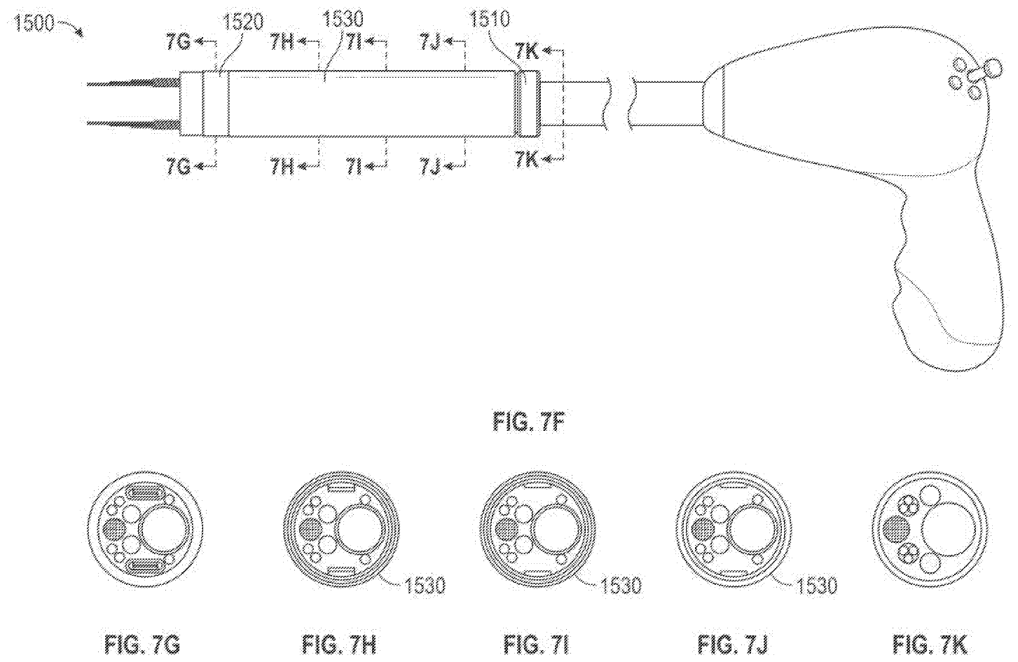

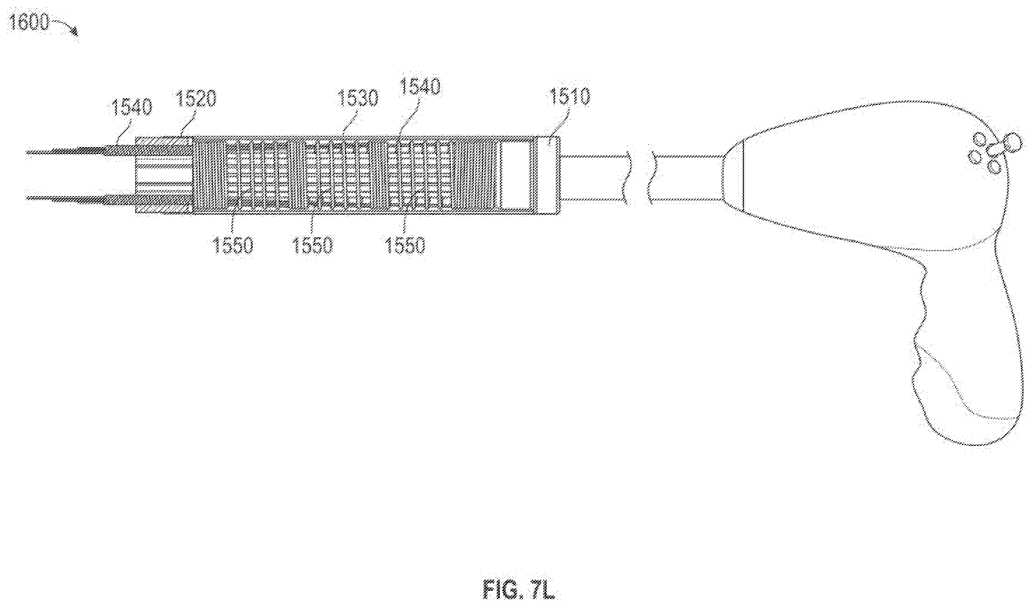

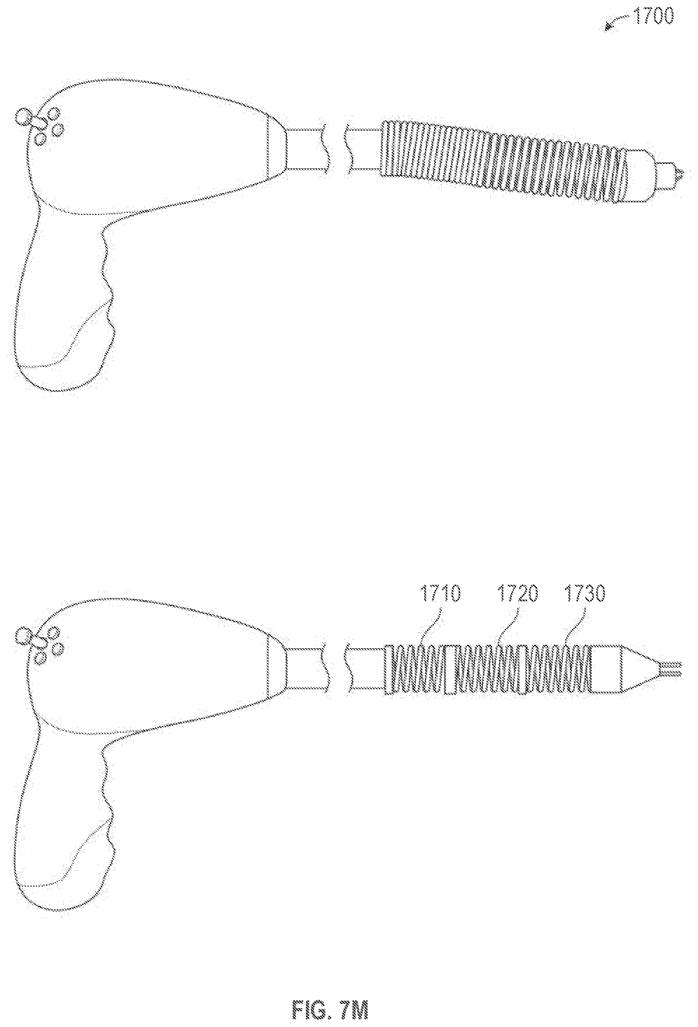

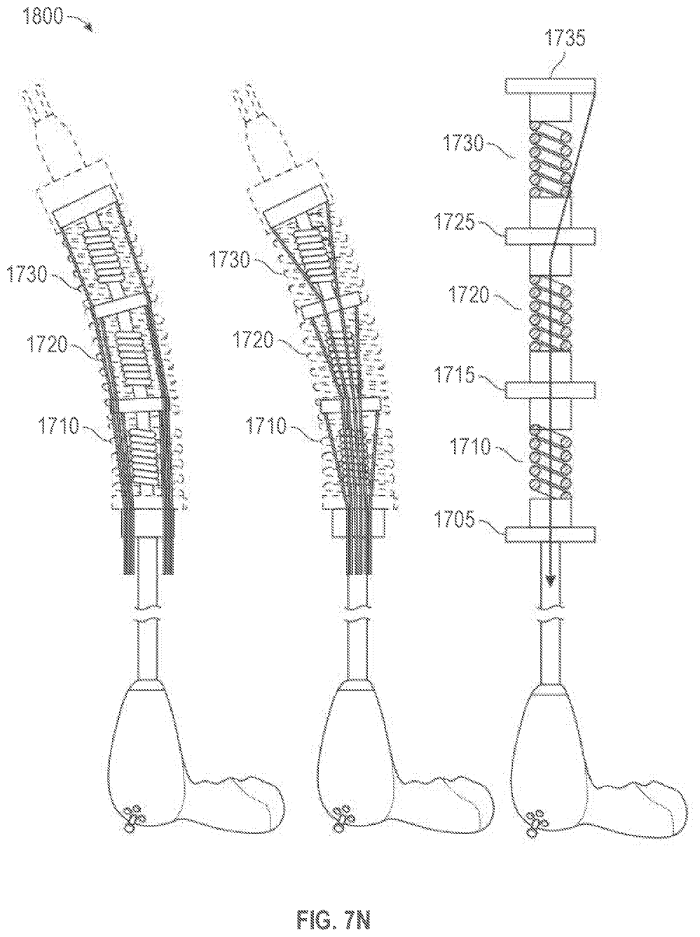

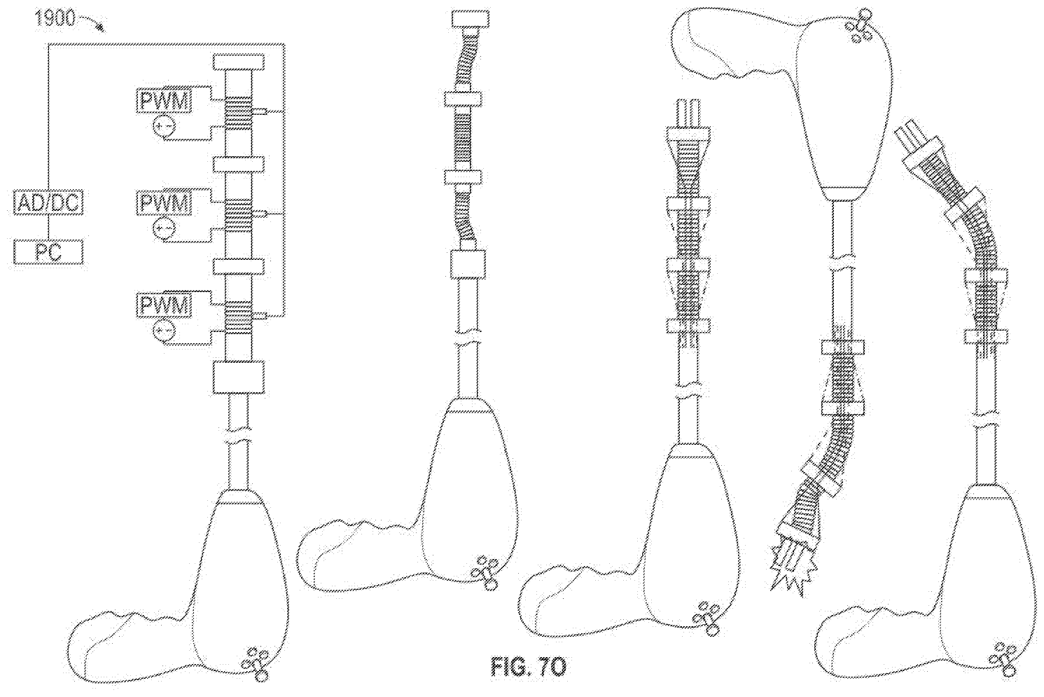

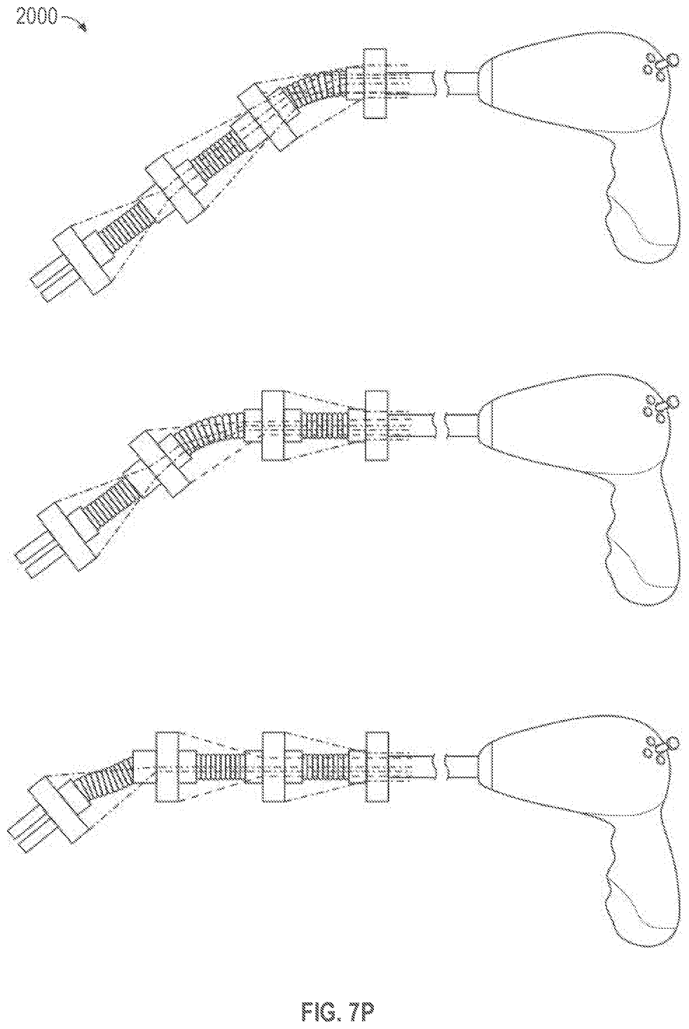

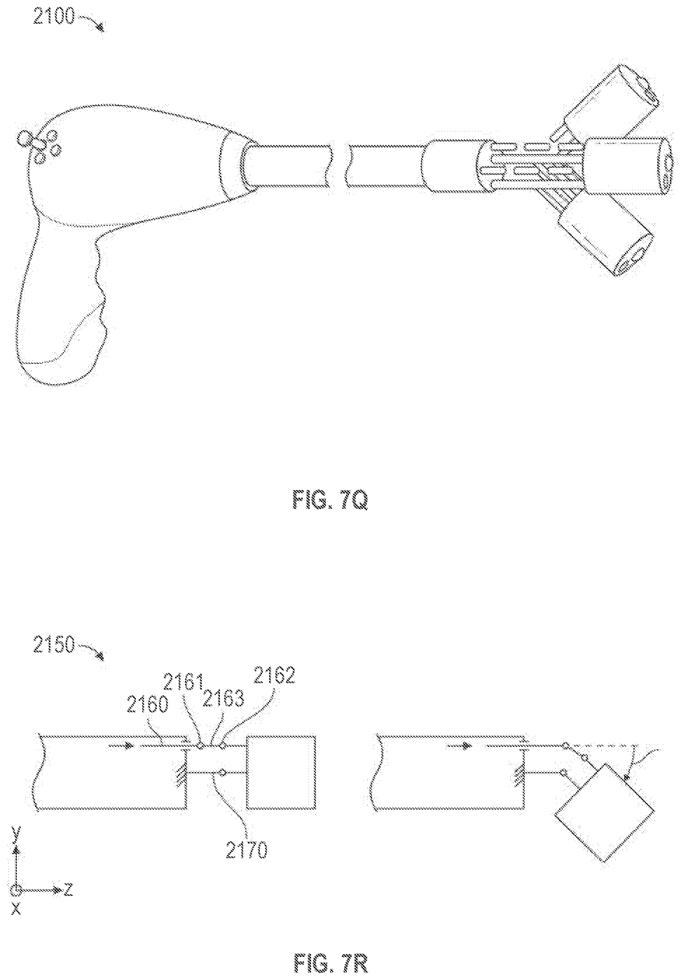

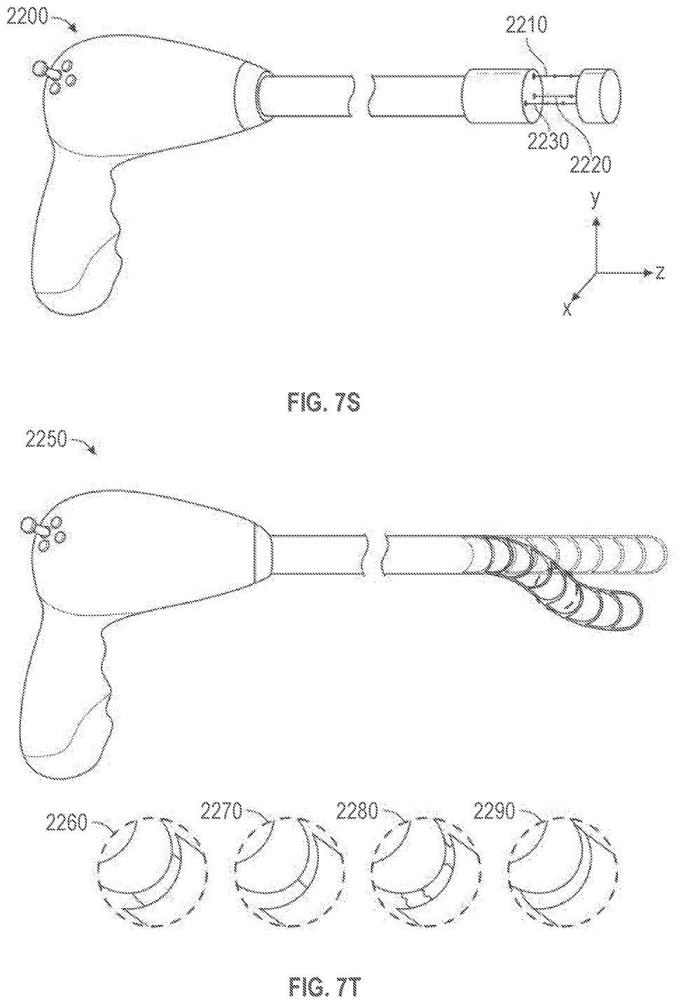

In various embodiments of the figures, a cutaway portion is shown that provides a zoomed view of the articulating tip of the laparoscope and does not indicate any break in structure of the laparoscope. The cutaway portion showing the zoomed in perspective of the articulating tip is not to scale with the other components of the laparoscope. Additionally, the cutaway portion shows internal components of the articulating tip of the laparoscope which are covered with medical grade hygienic wire sheathing to shield the components from the internal abdomen of a patient. See, for example, cutaway portions shown in FIGS. 7A, 7B, 7D, 7E, 7F, 7L, 7M, 7N, 7O, 7P, 7Q, 7R, 7S, and 7T. It should be understood that this sheathing is in place in various embodiments (even though it may not be shown clearly in the figure to further illustrate the internal components and embodiments of the articulating tip of the laparoscope).

Referring now to FIGS. 7A and 7B, there are shown a cross section (FIG. 7A) and a perspective view (FIG. 7B) of exemplary mechanisms 1000, 1100 for controlling movement of a tip. In implementations, wires (e.g., thick black curves in FIG. 7A) and pulleys to control the movement of endoscope tip, and there is least one lever member (1010 in FIG. 7A and FIG. 7B) arranged to be pivotable about a pivot axis, a pulley element located between the proximal end of the handle and the pivot axis of the lever member, and wherein the two control wires are attached to the at least one lever member, a first of said two control wires being arranged such that it travels from the at least one lever member in the direction towards the bending portion (the other end of the wire is attached (1120 in FIG. 7B) and the second of said two control wires being arranged such that it travels from the at least one lever member in the direction towards the pulley element, it then travels around the pulley element and then forwards towards the bending portion. The tip will bend toward the direction where one of the wires are being pulled by the lever allowing the tip of the laparoscope to move with up to and including 360-degrees of rotation.

Referring now to FIG. 7C, there is shown a perspective view of exemplary wire arrangements 1200 for controlling movement of a tip. In various implementations, tension wires (1210, 1220, 1230, 1240 in FIG. 7C) may be used in the main rod (lumen, 1250 in FIG. 7C) to move the tip in various directions as depicted (i.e., +x, −x, +y, −y, +z, and −z directions) allowing the tip of the laparoscope to move with up to and including 360-degrees of rotation.

Referring now to FIG. 7D, there is shown a side view of exemplary mechanisms 1300 for controlling movement of a tip. In various implementations, an arrangement of tension wires may be used to control the movement of the tip. A device may include the lens/camera tip 1330, the control unit 1310, and a control handle 1320. Manipulating the control unit 1310 and/or the handle 1320 actuates the lens/camera tip 1330 in various directions according to various degrees. For example, by pushing the control unit 1310 and/or the handle 1320 to the left, the tip will bend downward, and by pushing the control unit 1310 and/or the handle 1320 to the right, the tip will bend upward. According to various embodiments, the control unit 1310 and/or the handle 1320 may be positioned adjacent to either a joystick and/or buttons and/or a touchscreen on the control panel.

Referring now to FIGS. 7E, 7F, and 7G-7K, there are shown side breakaway views (FIG. 7E), a side view (FIG. 7F), and cross-sectional views (FIGS. 7G-7K) of exemplary mechanisms 1400, 1500 for controlling movement of a tip. In various implementations, the arrangement of the wires in the control unit may be as depicted in FIG. 7E. Moving the handle (1410) will mechanically pull and push wires leading to movement of the tip. The arrangement of wires in the main rod may be as depicted in FIGS. 7F, 7G, 7H, 7I, 7J, 7K, and/or 7L. One end of the wires is attached to the inside of the viewing tip end 1510. The outer covering (1500, 1600) preferably is constructed of a thin elastomeric material for maximum flexibility. At the proximal end of the controllably bendable section (1500, 1600), a stainless steel coupling sleeve 1520 extends between the outer covering 1530 and the coil spring 1540 and thereafter bonds to the outer co-extrusion of the insertion tube 1540. The coupling sleeve 1520 may be glued, soldered, brazed or epoxied to the coil spring 1540, and glued or epoxied to the covering 1530 and outside of the tube 1540. A second stainless steel coupling sleeve 1520 (see FIG. 7L) extends between the outer covering 1530 and the distal end of the coil spring 1540 and thereafter around a reduced portion of the viewing tip 1510. The sleeve 1520 may be glued, soldered, brazed or epoxied to the coil spring 1540 and viewing tip 1510, and glued or epoxied to the outer cover 1530. The resulting assembly comprises a chamber within the outer cover 1530 wherein the coil spring 1540 attaches at both ends yet is free to move axially in between. The resulting assembly when controlled by control unit allows the tip of the laparoscope to move with a maximum 360-degrees of rotation.

Referring now to FIG. 7L, there is shown a side breakaway view of exemplary mechanisms 1600 for controlling movement of a tip. In implementations, three springs 1550 may be used to build three joints. There may be wires (or tendons, instead of wires) to bend the joints. Bending may occur with multiple (e.g., three) joints simultaneously and/or at multiple (e.g., three) joints individually by pulling or pushing wires attached to the end of the each of three springs 1550. The resulting mechanism when controlled by control unit allows the tip of the laparoscope to move with the maximum 360-degrees of rotation.

Referring now to FIGS. 7M and 7N there are shown side breakaway views of exemplary mechanisms 1700, 1800 for controlling movement of a tip. In implementations, there may be three sets (e.g., red, blue, and orange) of wires to make bending at three joints 1710, 1720, 1730 (or springs 1710, 1720, 1730). There may be two possible wire (or tendon) connections. (i) peripheral wire routing. (ii) central wire routing. (iii) end segment actuation for motion in one direction where a wire branches out at disk 1725 and terminates at disk 1735 in FIG. 7N. In implementations, there may be three possible bending locations 1710, 1720, 1730 positioned between disks 1705, 1715, 1725, and 1735. In FIG. 7N, by pulling the left wire out of the longest ones, the last joint 1730 will bend to the left. By pulling the left wire out of the mid length ones, the second joint 1720 will bend to the left. By pulling the left wire out of the shortest wires, the first joint 1710 will bend to the left. The resulting mechanism when controlled by control unit allows the tip of the laparoscope to move with a maximum 360-degrees of rotation.

Referring now to FIGS. 7O and 7P, there are shown side breakaway views of exemplary mechanisms 1900 and 2000 for controlling movement of a tip. In implementations, a moveable tip may be at any of three angle positions. There may be three steering thin rods in the main rod. Two of the steering thin rods may perform a longitudinal movement driven by motorized spindles. The motors and the spindles can be positioned in the handle. One of the thin rods may be maintained as stationary at the shaft. There is a hinge on this rod which buckles due to the longitudinal translation of one or both moving rods.

Referring now to FIGS. 7Q, 7R, and 7S, there are shown side views of exemplary mechanisms 2100, 2150 and 2200 for controlling movement of a tip. FIG. 7Q shows the angles of tip movement by pulling and pushing thin rods. In implementations, the longitudinal travel of thin rod 2160 leads to the buckling of the hinges 2161 and 2162 separated by a space 2163. The stationary rod fixed at a distance 2164 as well as the length of the spacer 2163 determine the size of the bending angle α for a given movement of thin rod 2160. To bend the tip in −y direction as can be seen in FIG. 7R (right), rod 2160 is pushed rightward toward the tip, and the tip will bend to downward (i.e., −y direction). To make the tip bend in +y direction, rod 2160 is pulled leftward away from the tip. To bend the tip in −x direction, rod 2230 as can be seen in FIG. 7S is pushed toward the tip, and the tip will bend in −x direction). To make the tip bend in +x direction, rod 2230 is pulled away from the tip. The resulting mechanism when controlled by control unit allows the tip of the laparoscope to move with a maximum 360-degrees of rotation.

Referring now to FIGS. 7S and 7T, there are shown a side view (top) and close-up views (bottom) of exemplary mechanisms 2200 and 2250 for controlling movement of a tip. In implementations, various stiffnesses of materials may be used in various ways. One way is to build the joint area (bending location). The tension of several wires or rods (2210 or 2230) or a central wire (2220) is used to lock the segments and to induce a stiffness change. The segments can be beads, cylindrical elements connected by spherical joints or rigid cylindrical elements. The change of stiffness comes from the friction between the elements due to the tension in the wires. The tensioning of multiple wires is already used for tip control of medical devices or snake-like robots. In various implementations, the use of bellows-like segment connectors (2280) allows for locking the segment with a defined angle. The use of an inter-segment made of soft material (2290) can lead to a locking of the segments over the whole section. The stiffness of a multilayer structure (a rubber layer between two rigid plates) can be modified by its compression. A solution based on an endoskeleton-like structure is proposed herein. The endoskeleton-like structure is made of soft and rigid segments. A cable is tensioned which compresses the soft joints, increasing the lateral stiffness of the structure (as the compression of the soft joint decreases the effect of the moment on the bending of the structure). The resulting mechanism when controlled by control unit allows the tip of the laparoscope to move with a maximum 360-degrees of rotation.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way appreciably intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications can be referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present disclosure is not entitled to antedate such publication by virtue of prior present disclosure. Further, the dates of publication provided herein can be different from the actual publication dates, which can require independent confirmation.

The patentable scope of the present disclosure is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

Insofar as the description above and the accompanying drawing disclose any additional subject matter that is not within the scope of the claims below, the disclosures are not dedicated to the public and the right to file one or more applications to claims such additional disclosures is reserved.

The foregoing descriptions of specific embodiments of the present invention have been presented for purposes of illustration and description. They are not intended to be exhaustive or to limit the present invention to the precise forms disclosed, and modifications and variations are possible in view of the above teaching. The exemplary embodiment was chosen and described to best explain the principles of the present invention and its practical application, to thereby enable others skilled in the art to best utilize the present invention and its embodiments with modifications as suited to the use contemplated.

It is therefore submitted that the present invention has been shown and described in the most practical and exemplary embodiments. It should be recognized that departures may be made which fall within the scope of the invention. With respect to the description provided herein, it is submitted that the optimal features of the invention include variations in size, materials, shape, form, function and manner of operation, assembly, and use. All structures, functions, and relationships equivalent or essentially equivalent to those disclosed are intended to be encompassed by the present invention.

The following is claimed:

1. A device for laparoscopy, the device comprising:
a controller configured as an ergonomic handle; and
a camera rod attachable to the controller and having a flexible joint adjacent to a tip thereof, wherein the flexible joint provides a range of motion to the tip, and wherein the tip is configured to:
emit an incident light to illuminate an anatomy of a subject; and
receive a reflected light to image the anatomy of the subject.

2. The device of claim 1, wherein the incident light and the reflected light comprises visible light, ultraviolet (UV) light, or both.

3. The device of claim 1, wherein the range of motion is a maximum 360-degree range of motion.

4. The device of claim 2, wherein each of a pitch and a yaw of the tip of the camera rod includes the maximum 360-degree range of motion.

5. The device of claim 3, wherein an articulation of the camera rod by the controller moves the tip about the flexible joint for a pitch movement, a yaw movement, or both.

6. The device of claim 1, wherein the tip comprises a lens and the camera rod houses at least one optical fiber, wherein the lens and the optical fiber are configured to transmit the reflected light from the anatomy to an image sensor associated with the device, wherein the image sensor is configured to convert the reflected light to an electrical signal that is transmitted to a processor associated with the device to convert the electrical signal to an image for display.

7. The device of claim 1, wherein the camera rod houses at least one signal cable having a lens and an image sensor at the tip of the camera rod, wherein the lens and the image sensor are configured to convert the reflected light to an electrical signal and the signal cable is configured to transmit the electrical signal to a processor associated with the device to convert the electrical signal to an image for display.

8. A system for laparoscopy, the system comprising:
a device for laparoscopy, the device comprising:
a controller configured as an ergonomic handle; and
a camera rod attachable to the controller and having a flexible joint adjacent to a tip thereof, wherein the flexible joint provides a range of motion to the tip, and wherein the tip is configured to:
emit an incident light to illuminate an anatomy of a subject; and
receive a reflected light to image the anatomy of the subject;
an image sensor configured to receive the reflected light and transmit an electrical signal based on the reflected light;
a processor; and
a non-transitory machine-readable medium having instructions stored thereon which, when executed by the processor, configure the processor to:
receive the electrical signal and transmit an image signal to a display configured to depict an image of the anatomy of the subject based on the image signal.

9. The system of claim 8, wherein the incident light and the reflected light comprises visible light, ultraviolet (UV) light, or both.

10. The system of claim 8, wherein the range of motion is a maximum 360-degree range of motion of a pitch and a yaw of the tip of the camera rod, and wherein an articulation of the camera rod by the controller moves the tip about the flexible joint for a pitch movement, a yaw movement, or both.

11. The system of claim 8, wherein the processor and the non-transitory machine-readable medium are integral with the controller.

12. The system of claim 11, wherein the display is integral with the controller.

13. The system of claim 8, wherein the processor and the non-transitory machine-readable medium are integral with a computer, wherein the controller is operably connectable to the computer.

14. The system of claim 13, wherein the display is integral with or operably connectable to the computer.

15. The system of claim 8, wherein the tip comprises a lens and the camera rod houses at least one optical fiber, wherein the lens and the optical fiber are configured to transmit the reflected light from the anatomy to the image sensor.

16. The system of claim 8, wherein the camera rod houses at least one signal cable having a lens and the image sensor is positioned at the tip of the camera rod, wherein the lens and the image sensor are configured to convert the reflected light to the electrical signal and the signal cable is configured to transmit the electrical signal to the processor.

17. A controller for a device for laparoscopy, the controller comprising:
an ergonomic handle configured to be grasped single-handedly;
a mechanism associated with the ergonomic handle and configured to control a maximum 360-degree range of motion of a pitch and a yaw of a tip of a camera rod; and
a switch panel configured to be operated single-handedly, wherein the switch panel is operably connected to the mechanism to control the pitch and the yaw of the tip of the camera rod with movement of the mechanism.

18. The controller of claim 17, further comprising a display that is integral with the controller, positioned adjacent to the switch panel, and configured to depict images of an anatomy of a subject thereon.

19. The controller of claim 17, wherein the tip comprises a lens and the camera rod houses at least one optical fiber, wherein the lens and the optical fiber are configured to transmit the reflected light from an anatomy of a subject to an image sensor associated with the device, wherein the image sensor is configured to convert the reflected light to an electrical signal that is transmitted to a processor associated with the device to convert the electrical signal to an image for display.

20. The controller of claim 17, wherein the camera rod houses at least one signal cable having a lens and an image sensor at the tip of the camera rod, wherein the lens and the image sensor are configured to convert the reflected light to an electrical signal and the signal cable is configured to transmit the electrical signal to a processor associated with the device to convert the electrical signal to an image for display.
